(19) United States
(12) Patent Application Publication
(10) Pub. No.: US 2006/0217650 A1
(43) Pub. Date: Sep. 28, 2006

(54) O'Gara pelvic reduction clamp

(76) Inventor: Tadhg James O'Gara, Lynbrook, NY (US)

Correspondence Address: TADHG O'GARA, 199 WEBSTER STREET, WEST NEWTON, MA, 02465, US

(21) Appl. No.: 11/307,207

(22) Filed: Jan. 26, 2006

Related U.S. Application Data
(60) Provisional application No. 60/654,861, filed on Feb. 22, 2005.

(51) Int. Cl. A61F 5/00

(52) U.S. Cl. 602/32; 602/36; 602/19

(57) ABSTRACT

A pelvic fixation device which allows the three dimensional reduction of pelvic fractures, dislocations, or fracture dislocations. The device is adapted to attach to the anterior aspect of each hemipelvis. On one side it is devised to fixate the hemipelvis while on the other side it is adapted to provide a mechanism for distraction or compression and maintenance of this manipulative reduction force in three dimensions, especially in an anterior/posterior or vertical/inferior direction for a prolonged period of time. The device is adapted to be portable by its inclusion of a frame which can be placed beneath the patient or it can be applied in an operating room or emergency room and used as definitive or adjunctive treatment to other surgical procedures on the pelvic bone.

### FIELD OF THE INVENTION

The invention relates to an apparatus and method for the reduction and stabilization of a disrupted pelvic bone in three dimensions. Specifically, the invention relates to an apparatus and method of fixating one hemipelvis to a stationary object such as an operating room table or radiolucent board while at the same time, the invention includes a traction device which displaces and then holds the contralateral hemipelvis into a reduced position.

### DISCUSSION OF THE RELATED ART

Pelvic disruptions are generally the result of high energy trauma. The injury generally consists of a disassociation of one half of the pelvis (hemipelvis) from the other half and from the body's spine and sacrum. As the energy of the injury increases, the degree of displacement increases. Many times, the pelvic ring is opened or closed like a book from an anterior or lateral direction, and the posterior part of the pelvis remains intact so that this hinging can occur. These cases can be temporarily or definitively managed by external fixation. Most surgeons find it easier to apply an external fixator frame from an anterior direction. Many times the patients are sick and require subsequent access to the abdominal and thoracic area, making posterior application of frames dangerous. U.S. Pat. Nos. 4,361,144; 6,162,222; have described these external fixation devices whereby pins or bone screws are applied from an anterior direction into the iliac crest or pubic symphyseal areas.

However, sometimes the trauma is so great that the posterior structures in the pelvis are disrupted and a hemipelvis becomes sheared either backwards (posteriorly), vertically, flexed, extended, or a combination of all these. Because the posterior ligaments have been disrupted, there is no longer a hinge around which the traditional anteriorly applied external fixator can open or close the pelvic disruption. The traditional anteriorly applied external fixator's inability to maintain the reduction of the severe vertically or posteriorly sheared pelvic disruption is well documented in the biomechanical tests of Dr. Marvin Tile, published in Fractures of the Pelvis and Acetabulum. Because of this, these severe injuries have required open, surgical reduction when the patients have recovered somewhat from their life-threatening injuries. Open reduction, meaning cutting through the skin and muscle to obtain anatomic fixation of the bone, was required because the typical external fixation devices mentioned earlier are inadequate at treating the shearing type pelvic ring disruption. U.S. Pat. No. 4,815,455 specifically addresses this type of shearing injury. This device is applied to the posterior pelvis and allows a three dimensional reduction. Because the device is applied from posteriorly it involves the turning of the patient onto the abdomen, chest, and face thus obscuring the vital organs from immediate access.

This patent describes a device which solves the problems not addressed in the previous art. It is applied in an anterior direction making access to the abdomen, head, and thorax safe, yet on the other hand it allows a three dimensional reduction as in U.S. Pat. No. 4,815,555. The invention described in this patent also provides for a board or frame placed posteriorly to the patient as in U.S. Pat. No. 5,350,378 which would allow the patient to be transported. This board or frame however is not necessary for the unique function of the device as a whole, and the device can function well secured to an operating room table or other foundation.

### SUMMARY OF THE INVENTION

The invention is adapted to allow the three dimensional reduction of pelvic disruptions, especially the difficult sheared, high energy disruption. Unique to the design is its ability to be applied from an anterior direction, thereby minimizing the risk to the already sick patient by providing the trauma team continued access to the head, thorax, and abdomen. The invention can manipulate and then hold the pelvis in a reduced position while allowing the simultaneous use of xray guidance for further surgical procedures on the pelvis. The patent covers any embodiment of the described invention in regard to the material used for its construction including but not limited to carbon bars, metal, metal alloys, plastics, or radiolucent materials. The patent also describes a device which can be disposable or reusable.

The device consists of two parts. The first part or Part A, involves the method and device for the attachment of one hemipelvis to either the operating room table, a board, or any other area from which a stable foundation can be obtained. The second part or Part B, involves the method and device for the manipulation of the opposite hemipelvis. The device Part B, further includes both a frame for the pelvis's attachment to a stanchion device or lever arm and the stanchion device or lever arm itself from which traction can be applied in multiple planes. The stanchion device or lever arm is in turn secured to either the operating room table, the floor, a large board, or to any other position from which a secure foundation can be obtained. The stanchion device or lever arm provides a means of delivering traction in a multitude of planes, adjustable by the operator.

The device Part A, includes a plurality of threaded pins or bone clamp device, inserted or applied into or onto the anterior aspect of the pelvis. The device Part A further includes a frame which secures to the threaded pins or bone clamp at its one end and secures itself to a solid foundation at its other end. The device Part A can be secured to a solid foundation such as an operating room table, the floor, or the ceiling, and the device Part A incorporates this frame as well as any clamp, vice, or mechanism which can secure said frame to any potentially solid foundation. The device Part A also can include a large board or frame which is placed beneath the patient to which the frame securing the threaded pins into the anterior pelvic bone is attached.

The device Part B includes a plurality of threaded pins or bone clamp devices, inserted or applied into or onto the anterior aspect of the other, contralateral pelvic bone. The device Part B further includes a frame which secures to the shanks of the threaded pins or bone clamping devices. The frame also includes a part to which a wire or bar can be attached and upon which traction can be delivered to the pelvic bone. The attachment point for this wire or bar is adjustable and therefore the level arm's length is variable and adjustable which can thereby deliver a torque. The device Part B further includes a wire or bar or any other device or method of delivering traction to the frame. The device Part B further includes the stanchion support or lever arm for this wire or bar or traction delivery device and the stanchion support or lever arm is attached to a secure foundation which can include the operating room table, the floor, the ceiling, or the large board or frame beneath the patient as mentioned in Part A.

The device is operated as follows: The plurality of screws or bone clamping device mentioned in Part A and Part B are inserted or attached into or onto the anterior aspect of each hemipelvis. This plurality of screws can include screws into the Iliac wing, the anterior superior iliac spine, the anterior inferior iliac spine, or a transfixion screw directed posteriorly from the anterior pelvis through the ilium and out through the posterior aspect of the pelvis or a bone clamping device which attaches to the anterior pelvic ring in a clamping manner. In one hemipelvis this plurality of screws or bone clamping device are then secured to Part A of the device and Part A of the device is in turn secured to a stationary foundation or to a large frame or board placed beneath the patient. The opposite hemipelvis's plurality of screws or bone clamping devices are then attached to the frame Part B. The stanchion device or lever arm of Part B is separately secured to a solid foundation or to the frame or board placed beneath the patient. A wire or bar connector which can apply traction is then connected between the stanchion device or lever arm and the frame secured to the plurality of screws in the anterior pelvis of Part B. The exact point of attachment of this wire or bar onto the frame is adjustable by the operator so that traction can not only deliver distraction force but also a rotational torque vector dependent on the distance the operator attached the wire from the frame/pelvis unit's center of mass. The operator then adjusts the amount of traction delivered to the pelvis. This invention allows for the use of traction from any method including but not limited to an electrical motor, a hydraulic device, an operator powered crank or mechanical device, or simply by the operator pulling on the traction wire, rope, or bar and then locking the device after the traction had been delivered.

An exemplary embodiment of the invention is demonstrated via the following drawings.

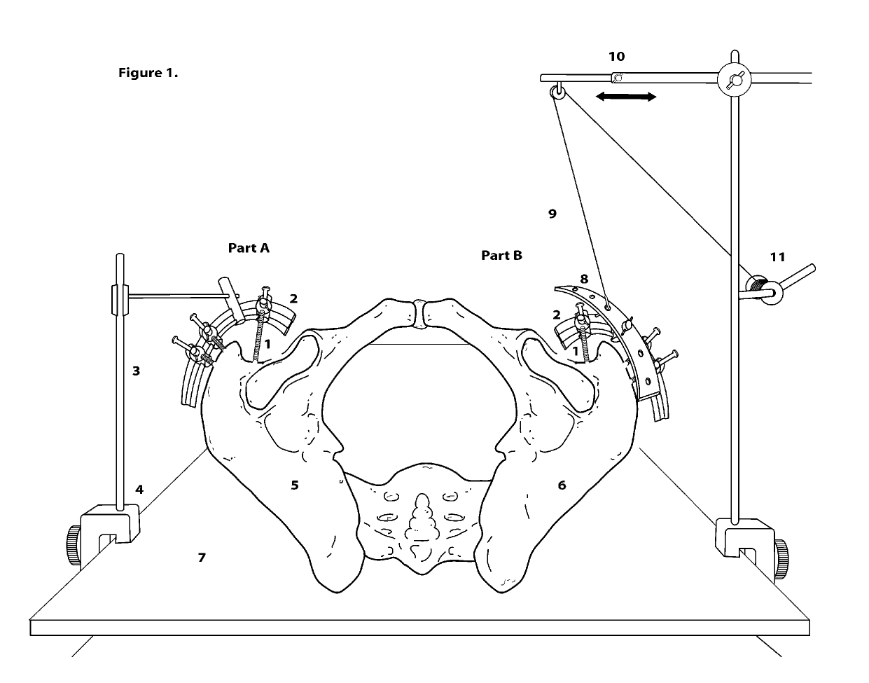

FIG. 1 illustrates both Part A and Part B which are anchored to the pelvis via anterior fixation screws. Note that Part A fixates one half of the pelvis to the construct beneath the pelvis while Part B includes a method of delivering traction in a vertical direction, thus providing a movement force vector between each half of the pelvis.

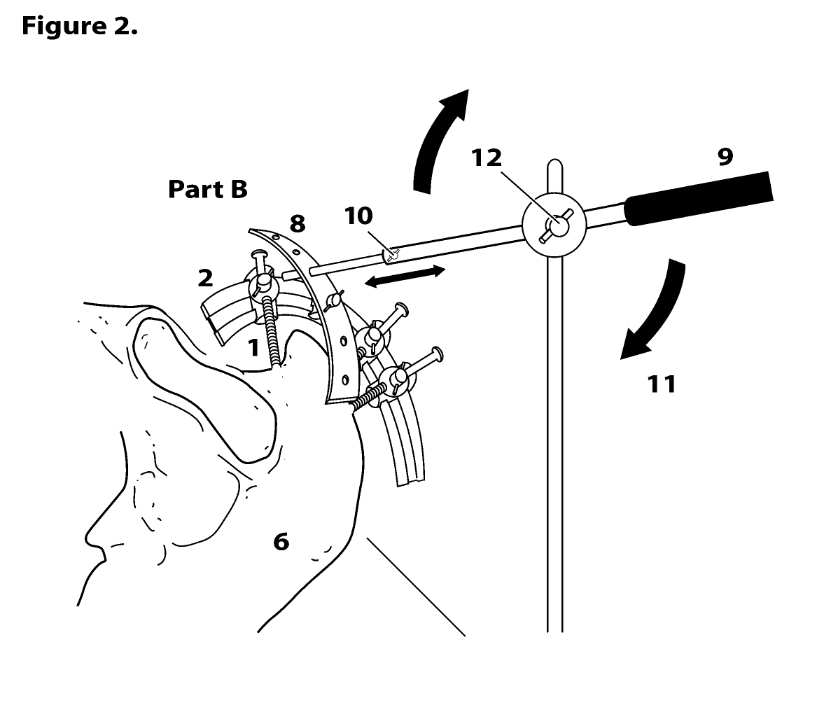

FIG. 2 is another exemplary embodiment in which Part B is including a simple mechanical arm to deliver traction instead of stanchion arm.

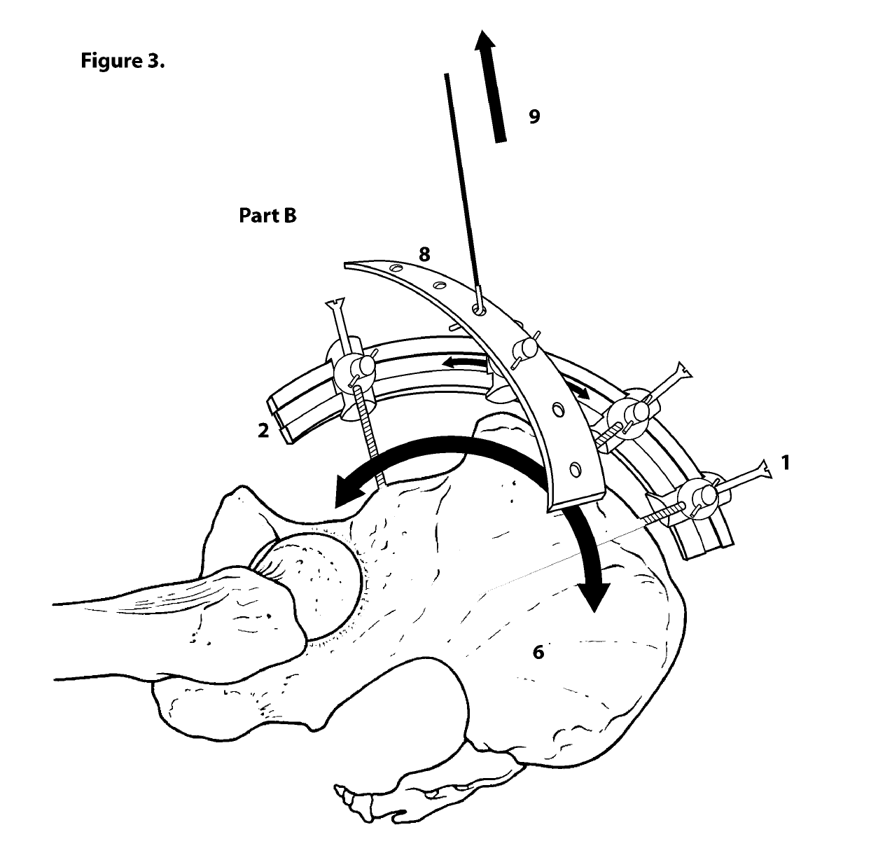

FIG. 3 is a side view of Part B, representing an exemplary embodiment of a frame which allows variability in the point of influence of the traction force.

Of note in this invention and the exemplary embodiments illustrated here is that various equivalent changes made to the design will fall within the invention idea. What is unique to the invention is its ability to apply distractive forces in 3 dimensions between halves of a pelvis via a frame attached from an anterior direction. For this to occur there are several exemplary embodiments noted herein which are necessary for the invention to function as described.

Firstly, In FIGS. 1 and 2, 1 demonstrates bone screws which are secured into the anterior aspect of the pelvis however the invention will also encompass vice-like clamps which could be applied to clamp onto the subcutaneous bone. 2 is a frame which secures all the screws or bone clamps together to form a solid unit and 3 is a simple bar which secures the frame 2 and screws or bone clamps 1 to the secure base 7 via a clamp 4. This composes Part A which is unique in that it has secured hemipelvis 5 to the secure base 7 via an anterior insertion of bone screws or application of bone clamps.

Secondly unique to the invention is its ability to enact a movement force between hemipelvis's 5 and 6 as seen in FIGS. 1 and 2. Since hemipelvis 5 is fixed via Part A (described above) to a secure base 7, the invention Part B will encompass any device which can deliver this movement force via its attachment anteriorly either via bone screws 1 or bone clamp described previously. Part B in FIGS. 1, 2 and 3 demonstrates also a frame 2 which secures the bone screws 1 or clamps. FIG. 1 demonstrates the force transmitter, an example here illustrated as a wire 9 suspended from a stanchion arm whose position above pelvis is variable as seen by 10 and whose power is run by a winch device 11. Another exemplary embodiment of the force transmitter is illustrated in FIG. 2 where the traction delivery device is a simple lever arm 9 which is also adjustable in its length 10 and whose force could be generated manually by pulling down on a lever arm 11 and then locked in position by tightening a locking bolt 12 thus maintaining the force.

FIG. 3 demonstrates a side view of Part B where 8 exemplifies a frame attachment which is adjustable to many positions on frame 2 and whose purpose is to vary the moment arm over which the force transmitter 9 acts. This in turn will provide either flexion, extension, vertical translation, anterior translation, internal or external rotational vectors to the hemipelvis 6 dependent on the position of force transmitter 9's attachment to the pelvis. This attachment is precisely varied by 8's attachment to 9 and in turn by 8's attachment along an adjustable rail on frame 2. The embodiment seen as frames 2 and 8 illustrate an example of an invention which also includes any method of adjusting the moment arm between a force transmitter exemplified as 9 and the composite center of mass of the hemipelvis 6 and bone attachment screws or clamps exemplified as 1. In other words, any type of frame which secures itself either directly or indirectly to the anterior pelvis and then which provides a means of securing a traction or force delivery transmitter to a position on itself which is variable by the operator is included within the scope of the invention.

What is claimed is:

1. A device for enabling the anatomic reduction of pelvic fractures, dislocations, or fracture dislocations in 3 planes comprising:
a) a first part, securing anteriorly to one hemipelvis and providing means of fixating it to a stationary object, thereby stabilizing one half of pelvic ring;
b) a second part, having two points of fixation, the first of which secures anteriorly to the other hemipelvis, the other half securing to a stationary object, and between providing the mechanism for delivering traction directly to pelvic ring via a stanchion device or lever arm or any method of force vector application through said stanchion device or lever arm which is fixated to a stationary object, as well as mechanism that enables variation of position and direction of said force vector upon the part secured to the bone;
c) a frame beneath patient which can take the place of described stationary objects and to which both parts of described invention can be secured.

2. A device as in claim 1, where the two parts of device are secured to pelvis either via threaded bone screws, the shanks of which provide for attachment of the device's two parts or via a bone holding clamp which attaches itself to the anterior aspect of the pelvis.

3. A device as in claim 1 a whereby pelvis is fixated to stationary object via frame attaching on one end to shanks of bone screws or bone holding clamp and on other end via clamp or vise-like attachment to stationary object such as operating room table or frame or board beneath patient.

4. A device as in claim 1b comprising a frame which attaches to bone screw shanks or bone holding clamps and upon which the traction method acts and whereby the location of the traction method's point of influence can be adjusted in multiple planes.

5. A device as in claim 1b, part of which comprises a wire or bar or lever arm or other such means of transmitting traction, attached on one end to stanchion device or force generator and on other end to frame described in claim 4.

6. A device as in claim 1b, part of which comprises a stanchion device or lever arm providing a position of leverage and a mechanism of generating and delivering force, providing a position of leverage which is adjustable and a method of maintaining traction through the device described in claim 5, such methods including but not limited to electric motor, mechanical device, hydraulic device, operator power, or any other method of delivering and maintaining force.

7. A device as in claim 6 which includes at one end of described stanchion device or lever arm force generator, a clamp or attachment method to secure said device to floor, ceiling, operating room table or frame beneath patient and which may also contribute to adjustable position of lever arm delivering traction to pelvis.

8. A device as in claim 1c which can be placed beneath patient and to which the device described in claim 1a and claim 1b are easily attachable and can thereby provide means of transporting patient with pelvic reduction maintained.
